(19) United States
(12) Patent Application Publication
(10) Pub. No.: US 2025/0025603 A1
(43) Pub. Date: Jan. 23, 2025

(54) ISOTROPIC SURGICAL IMPLANTS WITH FLOW-DEPOSITED CERAMIC PARTICLES

(71) Applicant: Orthomod LLC, Dayton, OH (US)

(72) Inventors: Shawn A. Hunter, Springboro, OH (US); David Louis Kirschman, Dayton, OH (US); Dewey J. Weeda, Oakwood, OH (US)

(73) Assignee: Orthomod LLC, Dayton, OH (US)

(21) Appl. No.: 18/778,077

(22) Filed: Jul. 19, 2024

Related U.S. Application Data
(60) Provisional application No. 63/514,376, filed on Jul. 19, 2023.

(51) Int. Cl. A61L 27/44; A61L 27/46; B29C 45/00; B29C 45/40; B29K 105/00; B29K 105/16; B29K 509/02; B29L 31/00

(52) U.S. Cl. CPC A61L 27/446; A61L 27/46; B29C 45/0013; B29C 45/0046; B29C 45/0053; B29C 45/40; A61L 2430/02; B29C 2045/0079; B29K 2105/0035; B29K 2105/16; B29K 2509/02; B29K 2995/0059; B29K 2995/007; B29K 2995/0094; B29L 2031/7532

(57) ABSTRACT

Provided herein are surgical implants and methods of manufacturing the implants. The surgical implant includes an outer body made of a polymer, at least one internal strut coupled to the outer body, where the internal strut has a smaller diameter than the outer body, and ceramic granules deposited on the surface of the internal strut. Methods of manufacturing a surgical implant generally include injecting a molten mixture containing a polymer and ceramic particles into a mold. The mold defines a negative space for forming the implant. As the molten mixture flows through the negative space, the mold constricts the flow at the internal strut, causing the ceramic granules to deposit on the surface of the internal strut. The mixture is then cooled to form the implant, with the ceramic granules at least partially exposed on its surface.

### CROSS-REFERENCES TO RELATED APPLICATIONS

This application is a continuation of United States Patent Application, which claims benefit of priority to U.S. Provisional Application No. 63/514,376, filed Jul. 19, 2023.

### TECHNICAL FIELD

The present disclosure relates generally to the field of surgical implants and methods of manufacturing the surgical implants, more specifically, to osteoactive surgical implants.

### BACKGROUND

Osteoconduction involves both the physical form of a surgical implant and the surface chemistry of the implant. Conventional implants relying on osteoactive ceramics like hydroxyapatite, beta-tricalcium phosphate, and silicates have limitations due to the brittle nature of these materials and issues with adhesion and delamination. Layer-based additive manufacturing techniques result in anisotropic implants with poor mechanical performance and difficulty in incorporating large ceramic granules effectively on the implant surface

Accordingly, a need exists for surgical implants and manufacturing methods that effectively incorporate osteoactive ceramic granules on the surface of the implant, while ensuring mechanical robustness and isotropic properties to enhance osteoconduction and overall implant performance.

### SUMMARY

This present disclosure relates to surgical implants and methods of manufacturing the implants.

In one embodiment, the present disclosure relates to a method of manufacturing a surgical implant. The method involves injecting a molten mixture containing a polymer and ceramic particles into a mold. The mold defines a negative space for forming the implant, which includes an outer body and at least one internal strut with a smaller diameter than the outer body. As the molten mixture flows through the negative space, the mold constricts the flow at the internal strut, causing the ceramic granules to deposit on its surface. The mixture is then cooled to form the implant, with the ceramic granules at least partially exposed on its surface.

In another embodiment, the present disclosure relates to a surgical implant. The surgical implant includes an outer body made of a polymer, at least one internal strut coupled to the outer body, where the internal strut has a smaller diameter than the outer body, and ceramic granules deposited on the surface of the internal strut.

Additional features and advantages of the embodiments described herein will be set forth in the detailed description that follows, and in part will be readily apparent to those skilled in the art from that description or recognized by practicing the embodiments described herein, including the detailed description that follows, the claims, as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE DRAWINGS

FIG. 1 illustrates an illustrative surgical implant of the present disclosure according to one or more embodiments described herein;

FIGS. 2A-2B illustrate an illustrative process of forming a surgical implant according to one or more embodiments described herein;

FIG. 3 illustrates a higher particle density and surface deposition at the transition from the implant outer body to the strut, according to one or more embodiments described herein; and

FIG. 4 illustrates the flow deposition of ceramic particles into internal struts, according to one or more embodiments described herein.

### DETAILED DESCRIPTION

The above described deficiencies are addressed by the present disclosure, which combines ceramics with high-strength carriers and/or substrates for use in load-bearing applications, such as in the musculoskeletal system of a subject.

Embodiments of the present disclosure generally relate to surgical implants and methods of manufacture. A method of manufacturing a surgical implant generally involves injecting a molten mixture containing a polymer and ceramic particles into a mold. The mold defines a negative space for forming the implant, which includes an outer body and at least one internal strut with a smaller diameter than the outer body. As the molten mixture flows through the negative space, the mold constricts the flow at the internal strut, causing the ceramic granules to deposit on its surface. The mixture is then cooled to form the implant, with the ceramic granules at least partially exposed on its surface. A surgical implant according to the present disclosure generally includes an outer body made of a polymer, at least one internal strut coupled to the outer body, where the internal strut has a smaller diameter than the outer body, and ceramic granules deposited on the surface of the internal strut.

As noted above, conventional osteoconductive ceramics are brittle and not compatible with the structural loads of the musculoskeletal system. Successful implants require a combination of mechanical strength and biological compatibility to support successful osteointegration and long-term functionality. Although, it may be theoretically possible to add ceramics to the conventional additive manufacturing techniques, introducing relatively large granules into an additive manufacturing process is technically difficult, inconsistent, and often produces poor outputs. Additionally, this addition would not result in an implant with a relative concentration of ceramics on the implant surface.

The disclosure should not be construed as limited to the embodiments set forth herein. Rather, these embodiments are provided so that this disclosure will be thorough and complete, and will fully convey the subject matter to those skilled in the art.

Unless otherwise defined, all technical and scientific terms used herein have the same meaning as commonly understood by one of ordinary skill in the art. The terminology used in the disclosure herein is for describing particular embodiments only and is not intended to be limiting.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

As used in the specification and the appended claims, the singular forms “a,” “an,” and “the” are intended to include the plural forms as well, unless the context clearly indicates otherwise. Thus, for example, reference to “a” component includes aspects having two or more such components, unless the context clearly indicates otherwise.

As used herein “osteointegration” or “osseointegration” and grammatical equivalents thereof refers to the process by which a surgical implant becomes anchored to the surrounding bone tissue. Osteointegration involves the direct structural and functional connection between the bone and the surface of the implant without the interposition of fibrous tissue. Successful osteointegration is characterized by the growth of new bone cells onto the surface of the implant, resulting in a stable and enduring bond that enhances the implant's stability and functionality.

As used herein “osteoconductive” and grammatical equivalents thereof refers to the ability of a material to serve as a scaffold or matrix that supports the growth and attachment of new bone cells. An osteoconductive material facilitates the migration, attachment, and proliferation of osteoblasts on its surface, promoting the formation of new bone tissue. For example and without being bound by theory, osteoconductive materials may create a conducive environment for bone regeneration by providing physical and chemical cues to support cellular activities related to bone formation.

As used herein “osteoactive” and grammatical equivalents thereof refers to the ability of a material to actively promote bone growth and regeneration by stimulating cellular activity. An osteoactive material may induce the formation of new bone through biochemical interactions with surrounding bone tissue. For example, and without being bound by theory, osteoactive materials may release ions or other bioactive molecules that can stimulate osteoblasts to proliferate, differentiate, and produce new bone matrix. Similarly, osteoactive materials may attract progenitor cells or stem cells to the site of implantation, promoting their differentiation into osteoblasts.

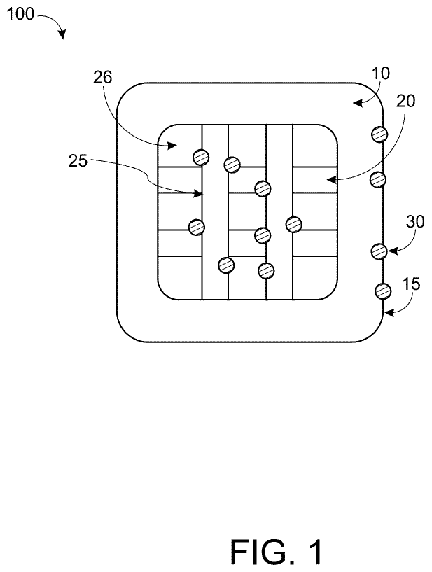

Referring now to FIG. 1, embodiments of the present disclosure generally include a surgical implant 100 comprising an outer body 10, at least one internal strut 20 coupled to the outer body 10, and ceramic granules 30. In some embodiments, the surgical implant 100 can be customized to meet the specific needs of a subject. This customization may include tailoring the size, shape, surface characteristics, lattice structure complexity, and/or materials to match the subject's anatomy and/or the clinical requirements of the procedure. For example, in some embodiments, the implantation site may be imaged, measured, and a mold, described in greater detail herein, may be rendered to produce a surgical implant, for example by 3D printing, that sizes and shapes the surgical implant to the subject's individual needs.

It will be appreciated that customization of the surgical implant 100 may be achieved through various techniques. For example, and without being bound by theory, in some embodiments, patient-specific imaging techniques such as CT scans, MRI, and 3D ultrasound may provide detailed anatomical data from the subject. This data may be used to generate 3D digital models for precise visualization and planning. In some embodiments, biomechanical analyses, including stress and strain assessments and finite element analysis (FEA) are performed to ensure that the surgical implant 100 withstands physiological loads. In some embodiments, described in greater detail herein, surface treatments, such as texturing, enhance tissue integration, while antibacterial and/or bioactive coatings may mitigate infection risks and promote healing. As described in greater detail herein, the lattice structure 25 may be adapted to adjust porosity, balancing strength with bone in-growth and reducing implant weight while maintaining structural integrity.

In some embodiments, the surgical implant 100 may include custom fixation features having specific attachment points and/or specialized anchoring mechanisms to ensure stability. In some embodiments, the surgical implant 100 is an adaptive design that incorporates bio-resorbable materials that dissolve as the body heals. Optionally, the surgical implant 100 includes systems for the controlled release of growth factors to enhance tissue regeneration.

It will be further appreciated that patient-specific surgical planning may employ software for preoperative simulations, to create custom surgical guises, and position the surgical implant 100 accurately. In some embodiments, customization of the surgical implant may be adapted based on machine learning algorithms or other artificial intelligence models.

It will be appreciated that the outer body 10 provides mechanical stability, strength, and a stable interface for osteointegration to the surgical implant 100. In some embodiments, the outer body 10 is formed to match the anatomical contours of an implantation site in a subject, thereby maximizing contact of the surgical implant 100 with surrounding bone.

In some embodiments, the outer body 10 of the surgical implant 100 comprises a biocompatible polymer suitable for a manufacturing process, described in greater detail herein. In some embodiments, the polymer is suitable for injection molding. Optionally, the polymer is a bioresorbable polymer. In some embodiments, the polymer is a non-bioresorbable polymer. In some embodiments, the polymer is a combination of bioresorbable and non-bioresorbable polymers.

As used herein, “bioresorbable polymers” refers to materials designed to degrade and be absorbed by the body over time. Exemplary bioresorbable polymers include, but are not limited to, polylactic acid (PLA), polyglycolic acid (PGA), polylactic-co-glycolic acid (PLGA), polycaprolactone (PCL), polyhydroxyalkanoates (PHA), polydioxanone (PDO), poly (trimethylene carbonate) (PTMC), poly (glycolide-co-caprolactone) (PGCL), poly (ethylene glycol) (PEG), copolymers thereof, combinations thereof, and the like. In some embodimetns, the polymer is polylactic acid. In some embodiments, the polymer is polyglycolic acid (PGA). In some embodiments, the polymer is polycaprolactone (PCL). In some embodiments, the polymer is poly (lactic-co-glycolic acid) (PLGA).

As used herein, “non-bioresorbable polymers” refers to materials that are not designed to degrade over time. Exemplary nonbioresorbable polymers include, but are not limited to, polyethylene (PE), polypropylene (PP), polytetrafluoroethylene (PTFE), polyetheretherketone (PEEK) polystyrene, polyvinyl chloride (PVC), polymethylmethacrylate (PMMA), acrylonitrile butadiene styrene (ABS), polycarbonate (PC), polyurethane, silicon rubber, nylon, copolymers thereof, combinations thereof, and the like. In some embodiments, the polymer is polyethylene (PE). In some embodiments, the polymer is polypropylene (PP). In some embodiments, the polymer is polyetheretherketone (PEEK). In some embodiments, the polymer is polymethyl methacrylate (PMMA).

As noted herein, in some embodiments, the polymer is a combination of one or more bioresorbable polymers and one or more non-bioresorbable polymers, forming a composite matrix. It will be appreciated that a composite matrix may be tailored to adjust the osteointegration rate of the implant, mechanical strength of the implant, or compatibility with the ceramic granules 30. For example, and without being bound by theory, polylactic acid (PLA) or poly (lactic-co-glycolic acid) (PLGA) may be combined with polyethylene (PE) or polyetheretherketone (PEEK) to achieve a balance between biodegradability and long-term stability. Bioresorbable components such as PLA or PLGA may facilitate gradual degradation and encourage new bone growth, while non-bioresorbable components like PEEK may provide sustained mechanical strength. Additionally, the incorporation of ceramic granules, such as hydroxyapatite or beta-tricalcium phosphate, into the composite matrix may enhance osteointegration and supply necessary structural support during the healing process.

Still referring to FIG. 1, the surgical implant 100 comprises at least one internal strut 20. Optionally, the strut(s) 20 are formed from the same material as the outer body 10. In some embodiments, the internal strut 20 promotes enhanced osteointegration and mechanical stability. An internal strut 20 may generally be a component that has an identifiable direction of greatest dimension and is coupled to the outer body 10 or to at least one other strut 20. In some embodiments, the internal strut 20 has a smaller diameter than the outer body 10. Optionally, the smaller diameter aids in flow constriction of the polymer during the injection molding process described in greater detail herein, thereby facilitating deposition of the ceramic granules 30.

The internal strut 20 may have two identifiable end points. In some embodiments, each endpoint is coupled to the outer body 10. In some embodiments, one endpoint is coupled to the outer body 10 and the other endpoint is coupled to another strut 20. In some embodiments, both endpoint are couple to another strut 20. Although the internal struts 20 depicted in the illustrations are substantially linear, any suitable shape of strut 20 is contemplated and possible. Optionally, the strut 20 has a substantially consistent cross-section across at least a portion of its length. In some embodiments, the internal strut 20 is at least partially exposed to an external environment along its complete circumference. Optionally, this exposure enables the osteoconductive ceramic granules 30 to interact with the surrounding bone tissue, thereby promoting osteointegration, described in greater detail herein.

In some embodiments, such as depicted in FIG. 1, the surgical implant 100 includes a plurality of internal struts 20. In some embodiments, the plurality of internal struts 20 can be arranged in a variety of configurations to provide structure and support to the surgical implant 100. It will be appreciated by those in the art that the arrangement of the plurality of struts 20 can offer mechanical benefits, increase surface area, enhance shock absorption, and/or reduce weight.

In some embodiments, such as shown in FIG. 1, the plurality of internal struts 20 can be arranged in a lattice structure 25, thereby creating an interconnected network within the implant 100. In some embodiments, the lattice structure 25 enables load distribution across the implant 100, thereby reducing the risk of mechanical failure from localized stress. The lattice structure 25 defines channels 26 within the surgical implant 100. These channels 26 provide a mechanism for osteointegration, allowing bone ingrowth and vascularization through the surgical implant 100.

In some embodiments, the lattice structure 25 provides a balance between mechanical strength and flexibility, allowing the surgical implant 100 to withstand physiological loads while promoting stress distribution throughout the surgical implant 100. It will be appreciated that the lattice structure 25 may vary in complexity from simple grids to more intricate geometries, depending on the specific application and mechanical requirements of the surgical implant 100. For example, and without being bound by theory, a denser lattice structure 25 may be used in load-bearing implants, while a more open lattice structure 25 may be used for implants requiring high levels of bone ingrowth.

In some embodiments, the outer body 10 and/or the internal strut 20 is substantially solid and/or isotropic. Optionally, the outer body 10 and/or the internal strut 20 is devoid of layer bonding or deposition that could create variable mechanical performance of the implant when tested in different axes. It will be appreciated that orthopedic implants created through 3D printing or other layer-based manufacturing processes can exhibit variable mechanical performance due to layer bonding, resulting in anisotropic properties when tested along different axes, as described in greater detail herein. In contrast, isotropic manufacturing processes, such as molding avoid this issue by producing homogeneous implants. For example, during the molding process described in greater detail herein, polymer material is injected into a mold cavity, ensuring distribution and solidification of the polymer without distinct layers. It will be appreciated that this homogeneous structure results in consistent mechanical properties in all directions, providing reliable strength and performance regardless of the testing axis. Consequently, molding techniques yield implants with more uniform mechanical characteristics, enhancing their effectiveness and reliability in clinical applications.

Optionally, the outer body 10 and/or the internal strut 20 comprises one or more surface characteristics that increases the surface area, thereby enhancing osteointegration and bone growth. In some embodiments, the outer body 10 and/or the internal strut 20 is porous to tailor osteointegration of the implant 100. As described in greater detail herein, this porosity may be achieved by introducing gas into the polymer during an injection molding process or by creating a porous structure through other additive manufacturing techniques. It will be appreciated that this porosity allows for bone ingrowth into the implant, further securing it in place and enhancing the biological integration with the surrounding tissue.

Optionally, the surface 15, (including a surface of the outer body 10 and/or a surface of the internal strut 20) of the surgical implant 100 is textured or roughened to enhance osteointegration. In some embodiments, described in greater detail herein, surface treatments such as chemical etching, supercritical fluid treatment, mechanical abrasion, and/or plasma spraying create microscopic features on the surface 15 that promote bone cell attachment and growth. In some embodiments, the surface 15 may include macroscopic features such as ridges, grooves, or porous coatings to promote bone anchorage.

In some embodiments, the surgical implant 100 may be stained with a dye to detect one or more surface characteristics on the surgical implant 100. In some embodiments, these surface characteristics and/or the presence of the ceramic granules 30, described in greater detail herein, may be detected with intermittent dye retention testing. As used herein, “intermittent dye retention” refers to the uneven or irregular absorption and retention of dye or stain on the surface 15 of the surgical implant 100. It will be appreciated that intermittent dye retention may occur when surface characteristics, such as texture, porosity, or composition, vary across different regions of the material, causing some areas to absorb more dye than others. In some embodiments, the surgical implant 100 demonstrates intermittent dye retention on the outer body 10 and/or the internal strut 20. Exemplary, non-limiting dyes include methylene blue, toluidine blue, alizarin red S, Indian ink, and/or rhodomine B. It will be appreciated that the dye or stain used for macroscopic intermittent dye retention is dependent on the specific application and the characteristics of the surgical implant 100. For example, and without being bound by theory, methylene blue and/or alizarin red S may be used for surgical implants with calcium and/or acidic components. Similarly, toluidine blue, Indian ink, and/or rhodamine B may be used in embodiments where detailed surface visualization is desired.

As noted above, the surgical implant 100 of the present disclosure also generally includes ceramic granules 30. Optionally, the ceramic granules 30 are more highly concentrated on the internal strut 20 due to flow constriction of the polymer matrix containing the ceramic granules 30 during the injection molding process, described in greater detail herein. In some embodiments, the composition, structure, and/or surface characteristics of the ceramic granules 30 support bone growth, provide mechanical reinforcement, and/or promote the bioactivity of the surgical implant 100.

Optionally, the ceramic granules 30 promote osteoconductivity and osteointegration. In embodiments, the ceramic granules 30 are osteoactive and/or osteoconductive. In some embodiments, the ceramic granules 30 contain one or more osteoconductive or osteoactive minerals. Illustrative examples include, but are not limited to, calcium and/or silicate compounds, such as monocalcium phosphate, tricalcium phosphate, hydroxyapatite, silicon dioxide, or bioglass.

In some embodiments, the ceramic granules 30 comprise an irregular outer surface. Optionally, the irregular outer surface increases the surface area of the ceramic granules 30, thereby promoting their interaction with the surrounding bone tissue at the implantation site. In some embodiments, the irregular outer surface promotes mechanical interlocking with the polymer matrix. In some embodiments, the ceramic granules 30 are porous, further increasing their surface area and providing additional sites for cellular attachment and bone ingrowth. Optionally, this porosity enhances the ceramic granules' 30 ability to release ions that promote osteogenesis.

In some embodiments, the ceramic granules 30 are microparticles. Optionally, the ceramic granules 30 range from about 50 μm to about 500 μm in diameter, including about 50 μm, about 60 μm, about 70 μm, about 80 μm, about 90 μm, about 100 μm, about 125 μm, about 150μm, about 175 μm, about 200 μm, about 225 μm, about 250 μm, about 275 μm, about 300 μm, about 325 μm, about 350 μm, about 375 μm, about 400 μm about 425 μm, about 450 μm, about 475 μm, about 500 μm, any range having endpoints defined by any two of the aforementioned values, and any subranges thereof. In some embodiments, the ceramic granules 30 have a diameter of from about 100 μm to about 300 μm.

In some embodiments, the ceramic granules 30 include microparticle ceramic granules and nanoparticle ceramic granules. Optionally, the nanoparticle ceramic granules may be used to coat the microparticle granules. In some embodiments, the nanoparticle ceramic granules are dispersed within the polymer matrix, described in greater detail herein.

In some embodiments, the ceramic granules 30 are at least partially exposed to an external environment. In some embodiments, the surgical implant 100 includes irregularly-shaped ceramic granules 30 partially protruding through, but anchored in, the surface 15 of the surgical implant 100 via one or more polymers. In some embodiments, the surgical implant 100 comprises a substantially regular polymer surface 15 interspersed with suspended, irregularly-shaped ceramic granules 30 protruding through the polymer surface 15. For example, in some embodiments, the ceramic granules 30 may partially protrude through the polymer surface 15 of the outer body 10 and/or the internal struts 20 creating a rough texture that further promotes cellular attachment and bone integration. In some embodiments, the ceramic granules' 30 exposure at the surface 15 allows for direct interaction with the biological environment, facilitating the release of ions that promote bone growth and healing. In some embodiments, the partially exposed ceramic granules 30 cause macroscopic intermittent dye retention of the surgical implant 100 as described herein.

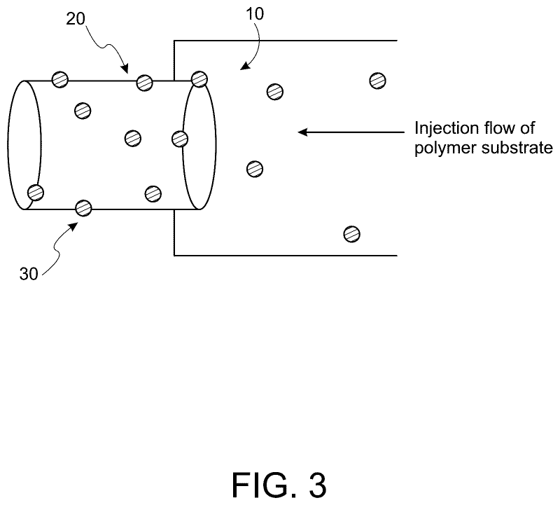

In some embodiments, the ceramic granules 30 are disposed at a higher density along the internal struts compared to the outer body 10, such as shown in FIG. 3. As described in greater detail herein, the ceramic granules 30 are deposited internally within the outer body 10 and/or the internal struts 20 due to the flow constriction during the injection molding process. It will be appreciated that this deposition is not uniform but occurs in discrete patches where the flow is most restricted. In some embodiments, the ceramic granules 30 are positioned discretely along the internal struts 20. Optionally, the surgical implant 100 includes areas of high osteoconductivity/osteoactivity (i.e., having more ceramic granules 30) interspersed with polymer regions. The discrete patches of ceramic granules 30 provide localized sites for osteoblast attachment and proliferation.

Optionally, the osteoactivity and/or osteoconductivity of the surgical implant 100 is controlled by the amount of ceramic granules 30 in the surgical implant 100. It will be further appreciated that adjusting the amount of ceramic granules 30 impacts the overall mechanical properties (e.g., structural integrity, load distribution, etc.) of the surgical implant 100, as well as the structural features (i.e., distribution of the ceramic granules 30, exposure of the ceramic granules through the polymer surface). In some embodiments, the ceramic granules 30 are less than 50% of the surgical implant 100 by weight. In some embodiments, the ceramic granules 30 are less than 40% of the surgical implant 100 by weight. In some embodiments, the ceramic granules 30 are less than 30% of the surgical implant 100 by weight. For example, and without being bound by theory, by adjusting the amount of ceramic granules 30, the surgical implant 100 promotes healthy bone integration while minimizing the risk of inflammation or other negative reactions.

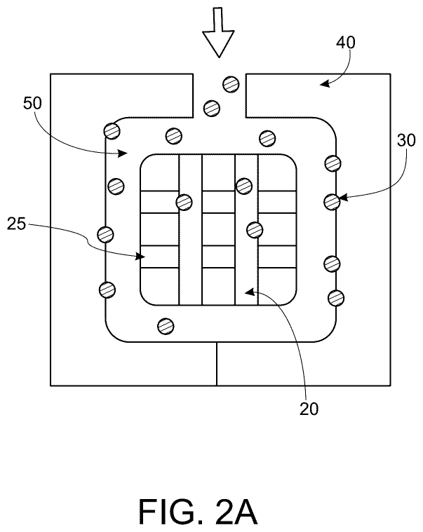

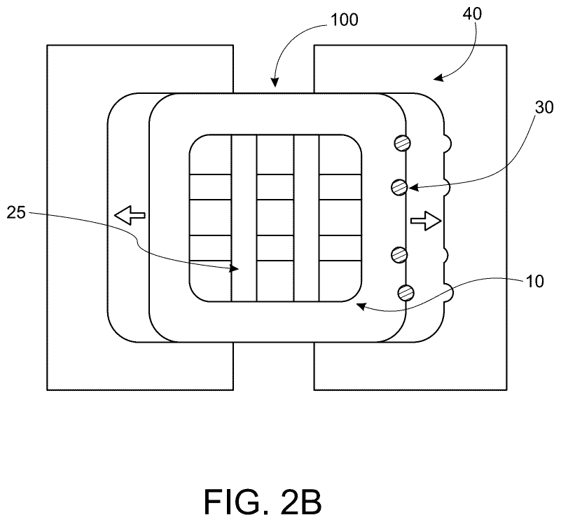

Turning now to FIGS. 2A-2B, the present disclosure also relates to methods of manufacturing the surgical implant 100. In some embodiments, the method of manufacturing the surgical implant 100 involves injecting a molten mixture containing the polymer and ceramic particles 30 into a mold 40. The mold 40 may generally define a negative space 50 for forming the surgical implant 100. In some embodiments, the molten mixture is injected into the negative space 50 and hardened to form the surgical implant 100. The surgical implant 100 may be as generally described herein. In some embodiments, the surgical implant 100 includes an outer body 10 and at least one internal strut 20 with a smaller diameter than the outer body. Optionally, as the molten mixture flows through the negative space 50, the mold 40 constricts the flow at the internal strut 20, causing the ceramic granules 30 to deposit on the surface of the internal strut 20. The mixture is then cooled to form the surgical implant 100, with the ceramic granules 30 at least partially exposed on the surface 15.

Optionally, the surgical implant 100 may be manufactured by injection molding. Other manufacturing processes or molding processes are contemplated and possible. In some embodiments, the surgical implant 100 is manufactured using an injection molding process where a mixture of polymer and ceramic granules is heated to a molten state and injected into a mold, described in greater detail herein.

In some embodiments, a mold 40 is designed with negative spaces 50 corresponding to the desired shape of the surgical implant 100, including the outer body 10, internal struts 20, and lattice structure 25. In some embodiments, the mold 40 is at least partially soluble. Optionally, the mold 40 may have soluble components and insoluble components, assembled into a cohesive mold 40. In some embodiments, the internal struts 20 and/or lattice structure 25 are formed via a soluble molding process, described in greater detail herein. In some embodiments, the mold 40 is a single-use, soluble mold that is later dissolved to reveal the surgical implant 100.

In some embodiments, the soluble mold 40 is dissolved partially or completely in a solvent without leaving a residue or affecting the integrity of the surgical implant 100. It will be appreciated that a soluble mold 40 enables the manufacture of surgical implants with intricate internal structures and lattice structure that would be difficult or impossible to achieve with traditional machining methods and eliminates the need for complex mechanical removal techniques, thereby reducing production time and costs.

In some embodiments, the mold 40 is formed from a deformable material that allows the ceramic granules to substantially protrude through the implant surface and indent the mold 40 without causing damage to the ceramic granules 30, as shown in FIG. 2B. In some embodiments, the mold 40 is formed from a material having a Brinell hardness of less than 15 HB. Exemplary, non-limiting materials that may be used to form the mold 40 include polyvinyl alcohol, polyethylene glycol, gelatin, starch-based polymers, methylcellulose, combinations thereof, and the like.

In some embodiments, a polymer powder is mixed with the ceramic granules 30 and compounded. Optionally, the mixture is fed into an extruder, where it is heated and combined to form a homogeneous mixture. In some embodiments, the extrusion process involves pushing the mixture through a die to create uniform polymer-ceramic strands. Optionally, the strands are cooled and pelletized to form pellets. In some embodiments, the pellets are heated to a molten state and injected into the negative space 50 of the mold 40 under high pressure.

In some embodiments, manufacturing the surgical implant includes gas incorporation to increase porosity of the implant. Optionally, gas is introduced to the molten mixture during extrusion or molding. In some embodiments, the gas is introduced to the molten mixture under pressure, which forms bubbles or voids within the polymer matrix as it solidifies. It will be appreciated that the size and distribution of these pores can be adjusted by varying the gas pressure and processing parameters.

Optionally, the flow dynamics within the mold 40 cause the ceramic granules 30 to be deposited at specific locations. In some embodiment, the flow dynamics are controlled by the design of the mold 40. In some embodiments, the mold 40 is configured to constrict flow at the internal struts 20. For example, and without being bound by theory, in some embodiments, constricting the flow involves narrowing the cross-sectional area of the mold 40 at strategic points, such as the internal struts 20. It will be appreciated that this narrowing increases the resistance to material flow, diverting more of the molten mixture towards the internal struts 20 rather than the outer regions of the mold 40 that form the outer body 10.

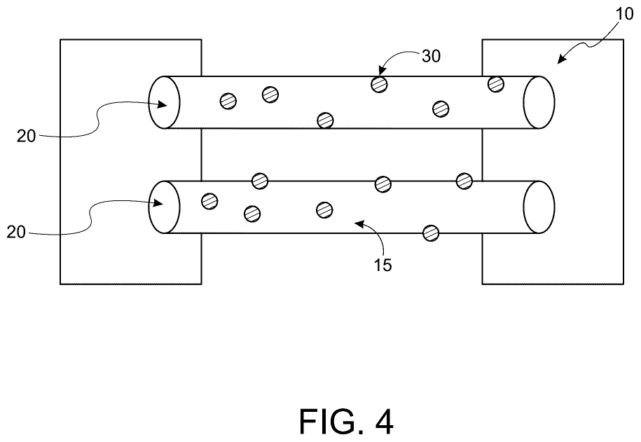

In some embodiments, as the molten polymer-ceramic mixture flows through the mold 40, the smaller diameter of the internal struts 20 creates a constriction. Optionally, this constriction deposits the ceramic granules along the surface 15 of the struts 20, such as shown in FIG. 4. In some embodiments, the flow constriction results in a higher density of ceramic granules 30 on the internal struts 20 compared to the outer body 10, where the flow is less restricted.

Once the molten polymer fills the mold 40, the polymer hardens into the surgical implant 100, as shown in FIG. 2B. In some embodiments, the surgical implant 100 is cooled in a controlled manner to solidify the material and ensure it retains the intended shape and structural integrity. Optionally, the molded implant is transferred to a cooling station or chamber where the temperature is gradually reduced. In some embodiments, the controlled cooling prevents the formation of internal stresses and defects in the surgical implant 100.

In some embodiments, the mold 40 is dissolved after the surgical implant is formed. In some embodiments, the molded surgical implant 100 is immersed in a solvent solution to dissolve the soluble polymer used in the mold 40. Optionally, the solvent is suitable for dissolving the mold 40 without affecting the structural integrity of the surgical implant 100, such as the ceramic granules 30 on the surface of the implant 100. In some embodiments, as the soluble polymer dissolves, the mold 40 leaves behind the complex internal structures and surface features of the surgical implant 100, such as the lattice structure 25 formed by the internal struts 20 and any intentional porosity created during the molding process.

Optionally, the surgical implant may undergo one or more post-processing steps. In some embodiments, the post-processing steps refine or enhance properties of the surgical implant 100. Exemplary post-processing steps include, but are not limited to, supercritical fluid treatment, chemical etching, mechanical abrasion, and/or annealing.

In some embodiments, the surgical implant 100 is treated with supercritical fluid to clean and/or modify the surface of the implant 100. Optionally, the surgical implant 100 is exposed to supercritical CO2, which penetrates the material and removes impurities without leaving any residue. In some embodiments, supercritical fluids may be used to introduce bioactive molecules or drugs onto the implant surface, enhancing its functionality and promoting better integration with surrounding tissues.

Optionally, the surgical implant 100 is chemically etched to introduce one or more surface characteristics described in greater detail herein. In some embodiments, the surgical implant 100 is immersed in a chemical solution that selectively removes material from its surface. In some embodiments, chemically etching the surgical implant 100 creates a rougher surface texture, thereby promoting osteointegration.

In some embodiments, the surgical implant 100 is mechanically abraded to alter the surface of the implant. Optionally, mechanical abrasion of the surgical implant 100 refines the surface texture to increase the osteointegration and/or osteoconductivity of the surgical implant 100. Exemplary mechanical abrasion techniques include grit blasting, sanding, polishing, and the like.

In some embodiments, the surgical implant 100 is annealed to increase the density of the ceramic granules 30 while maintaining the shape and surface characteristics. For example, and without being bound by theory, annealing the surgical implant may improve the polymer matrix's properties by relieving internal stresses and promoting molecular rearrangement and allows the polymer to better conform around the ceramic granules. This may improve the interface between the polymer matrix and the ceramic granules 30, thereby increasing the contact area and improving bonding.

Optionally, annealing the surgical implant 100 comprises applying a controlled heat treatment to the surgical implant 100 after the molding process. In some embodiments, the surgical implant 100 is heated to a temperature below the melting point of a polymer. Optionally, the temperature is high enough to allow molecular motion within the polymer matrix. In some embodiments, the polymer chains gain mobility during annealing and rearrange to relieve internal stresses and reduce voids or imperfections within the matrix.

In some embodiments, the annealing process results in increased density of the surgical implant 100. For example, in some embodiments, during the annealing process, the polymer chains become more ordered and aligned, the polymer matrix becomes denser, particularly around the embedded ceramic granules. It will be appreciated that this increased density may result from the polymer chains conforming more tightly around the granules, filling in any gaps and eliminating microvoids that may have been present after the initial molding process. It will be appreciated that, by closely conforming to the shape of the ceramic granules, the polymer matrix enhances the contact area between the polymer and the granules, leading to improved mechanical interlocking and bonding at the interface.

### Aspects Listing:

In a first aspect, alone or in combination with any other aspect described herein, the present disclosure relates to a surgical implant, comprising an outer body comprising a polymer; at least one internal strut, coupled to the outer body, wherein the at least one internal strut has a smaller diameter than the outer body; and ceramic granules deposited at a surface of the internal strut.

In a second aspect, alone or in combination with any other aspect described herein, the present disclosure relates to a surgical implant, wherein the ceramic granules are at least partially exposed at a surface of the surgical implant, such that staining of the surface reveals intermittent dye retention along the surface.

In a third aspect, alone or in combination with any other aspect described herein, the present disclosure relates to a surgical implant, wherein the stain is methylene blue.

In a fourth aspect, alone or in combination with any other aspect described herein, the present disclosure relates to a surgical implant, comprising a plurality of internal struts.

In a fifth aspect, alone or in combination with any other aspect described herein, the present disclosure relates to a surgical implant, wherein the plurality of internal struts form a lattice structure defining one or more channels through the implant.

In a sixth aspect, alone or in combination with any other aspect described herein, the present disclosure relates to a surgical implant, wherein the ceramic granules comprise an osteoconductive mineral.

In a seventh aspect, alone or in combination with any other aspect described herein, the present disclosure relates to a surgical implant, wherein the osteoconductive mineral comprises calcium or silicate.

In an eighth aspect, alone or in combination with any other aspect described herein, the present disclosure relates to a surgical implant, wherein the osteoconductive mineral comprises monocalcium phosphate, tricalcium phosphate, hydroxyapatite, silicon dioxide, or bioglass.

In a ninth aspect, alone or in combination with any other aspect described herein, the present disclosure relates to a surgical implant, wherein the ceramic granules comprise an irregular outer surface.

In a tenth aspect, alone or in combination with any other aspect described herein, the present disclosure relates to a surgical implant, wherein the ceramic granules comprise less than 30% of the surgical implant by weight.

In an eleventh aspect, alone or in combination with any other aspect described herein, the present disclosure relates to a surgical implant, wherein the ceramic granules are disposed at a greater density along the internal struts compared to the outer body.

In a twelfth aspect, alone or in combination with any other aspect described herein, the present disclosure relates to a surgical implant, wherein the surface comprises a substantially regular polymer surface interspersed with ceramic granules comprising substantially irregular outer surfaces.

In a thirteenth aspect, alone or in combination with any other aspect described herein, the present disclosure relates to a surgical implant, wherein the osteoactive ceramic granules have a diameter of from about 100 μm to about 300 μm.

In a fourteenth aspect, alone or in combination with any other aspect described herein, the present disclosure relates to a surgical implant, wherein the outer body and the at least on internal strut are substantially solid and isotropic in nature.

In a fifteenth aspect, alone or in combination with any other aspect described herein, the present disclosure relates to a surgical implant, wherein the outer body and the at least on internal strut do not contain layer bonding or layer deposition.

In a sixteenth aspect, alone or in combination with any other aspect described herein, the present disclosure relates to a surgical implant, wherein the ceramic granules are deposited at the surface of the internal strut during an injection molding process.

In a seventeenth aspect, alone or in combination with any other aspect described herein, the present disclosure relates to a method of manufacturing a surgical implant, the method comprising injecting a molten mixture comprising a polymer and ceramic particles into a mold, wherein the mold defines a negative space for forming the surgical implant; flowing the molten mixture through the negative space, wherein the mold is configured to constrict flow of the molten mixture at the internal strut, thereby depositing the ceramic granules at a surface of the internal strut; and cooling the molten mixture to form the surgical implant, wherein the ceramic granules are at least partially exposed at a surface of the surgical implant.

In an eighteenth aspect, alone or in combination with any other aspect described herein, the present disclosure relates to a method of manufacturing a surgical implant, further comprising staining the surgical implant with a dye to detect one or more surface characteristics on the surgical implant.

In a nineteenth aspect, alone or in combination with any other aspect described herein, the present disclosure relates to a method of manufacturing a surgical implant, wherein the dye is methylene blue.

In a twentieth aspect, alone or in combination with any other aspect described herein, the present disclosure relates to a method of manufacturing a surgical implant, wherein the surface characteristics comprise one or more of the ceramic granules, ridges, grooves, or pores

In a twenty-first aspect, alone or in combination with any other aspect described herein, the present disclosure relates to a method of manufacturing a surgical implant, wherein the mold is a soluble mold.

In a twenty-second aspect, alone or in combination with any other aspect described herein, the present disclosure relates to a method of manufacturing a surgical implant, wherein the mold is formed from a deformable material, thereby allowing the ceramic granules to protrude through the surface of the implant and indent the mold.

In a twenty-third aspect, alone or in combination with any other aspect described herein, the present disclosure relates to a method of manufacturing a surgical implant, wherein the mold has a Brinell hardness of less than 15 HB.

In a twenty-fourth aspect, alone or in combination with any other aspect described herein, the present disclosure relates to a method of manufacturing a surgical implant, wherein the ceramic granules are disposed at a greater density along the internal strut compared to the outer body.

In a twenty-fifth aspect, alone or in combination with any other aspect described herein, the present disclosure relates to a method of manufacturing a surgical implant, wherein the surgical implant is customized to meet the specific needs of a subject.

In a twenty-sixth aspect, alone or in combination with any other aspect described herein, the present disclosure relates to a method of manufacturing a surgical implant, wherein the polymer and ceramic particles are compounded during an extrusion process, pelletization process, molding process, or injection process.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. The term “substantially” is used herein also to represent the degree by which a quantitative representation may vary from a stated reference without resulting in a change in the basic function of the subject matter at issue. Thus, it is used to represent the inherent degree of uncertainty that may be attributed to any quantitative comparison, value, measurement, or other representation, referring to an arrangement of elements or features that, while in theory would be expected to exhibit exact correspondence or behavior, may in practice embody something less than exact.

Unless otherwise defined, all technical and scientific terms used herein have the same meaning as commonly understood by one of ordinary skill in the art to which the invention belongs. The terminology used in the description herein is for describing particular embodiments only and is not intended to be limiting. As used in the specification and appended claims, the singular forms “a,” “an,” and “the” are intended to include the plural forms as well, unless the context clearly indicates otherwise.

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that where a first component is described as “comprising” or “including” a second component, it is contemplated that, in some embodiments, the first component “consists” or “consists essentially of” the second component. Additionally, the term “consisting essentially of” is used in this disclosure to refer to quantitative values that do not materially affect the basic and novel characteristic(s) of the disclosure.

It should be understood that any two quantitative values assigned to a property or measurement may constitute a range of that property or measurement, and all combinations of ranges formed from all stated quantitative values of a given property or measurement are contemplated in this disclosure.

While particular embodiments have been illustrated and described herein, it should be understood that various other changes and modifications may be made without departing from scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A method of manufacturing a surgical implant, the method comprising:
injecting a molten mixture comprising a polymer and ceramic particles into a mold, wherein the mold defines a negative space for forming the surgical implant, the surgical implant comprising an outer body and at least one internal strut, wherein the at least one internal strut has a smaller diameter than the outer body;
flowing the molten mixture through the negative space, wherein the mold is configured to constrict flow of the molten mixture at the internal strut, thereby depositing the ceramic granules at a surface of the internal strut; and
cooling the molten mixture to form the surgical implant, wherein the ceramic granules are at least partially exposed at a surface of the surgical implant.

2. The method of claim 1, wherein the surgical implant comprises a plurality of internal struts.

3. The method of claim 2, wherein the plurality of internal struts form a lattice structure defining one or more channels through the implant.

4. The method of claim 1, further comprising staining the surgical implant with a dye to detect one or more surface characteristics on the surgical implant.

5. The method of claim 4, wherein the dye is methylene blue.

6. The method of claim 4, wherein the surface characteristics comprise one or more of the ceramic granules, ridges, grooves, or pores.

7. The method of claim 4, wherein the surgical implant demonstrates intermittent dye retention.

8. The method of claim 1, wherein the mold is a soluble mold.

9. The method of claim 1, wherein the mold is formed from a deformable material, thereby allowing the ceramic granules to protrude through the surface of the implant and indent the mold.

10. The method of claim 1, wherein the mold has a Brinell hardness of less than 15 HB.

11. The method of claim 1, wherein the ceramic granules comprise an osteoconductive mineral.

12. The method of claim 11, wherein the osteoconductive mineral comprises calcium or silicate.

13. The method of claim 11, wherein the osteoconductive mineral comprises monocalcium phosphate, tricalcium phosphate, hydroxyapatite, silicon dioxide, or bioglass.

14. The method of claim 1, wherein the ceramic granules are disposed are a greater density along the internal strut compared to the outer body.

15. The method of claim 1, wherein the surface of the surgical implant comprises a substantially regular polymer surface interspersed with ceramic granules comprising substantially irregular outer surfaces.

16. The method of claim 1, wherein the surgical implant is customized to meet one or more specific needs of a subject.

17. A surgical implant, comprising:
an outer body comprising a polymer;
at least one internal strut, coupled to the outer body, wherein the at least one internal strut has a smaller diameter than the outer body; and
ceramic granules deposited at a surface of the internal strut.

18. The surgical implant of claim 17, wherein the ceramic granules are at least partially exposed at a surface of the surgical implant.

19. The surgical implant of claim 17, comprising a plurality of internal struts.

20. The surgical implant of claim 19, wherein the plurality of internal struts form a lattice structure defining one or more channels through the implant.

21. The surgical implant of claim 17, wherein the ceramic granules comprise an osteoconductive mineral.

22. The surgical implant of claim 21, wherein the osteoconductive mineral comprises calcium or silicate.

23. The surgical implant of claim 21, wherein the osteoconductive mineral is selected from monocalcium phosphate, tricalcium phosphate, hydroxyapatite, silicon dioxide, or bioglass.

24. The surgical implant of claim 17, wherein the ceramic granules comprise an irregular outer surface.

25. The surgical implant of claim 17, wherein the ceramic granules comprise less than 30% of the surgical implant by weight.

26. The surgical implant of claim 17, wherein the ceramic granules are disposed at a greater density along the internal strut compared to the outer body.

27. The surgical implant of claim 18, wherein the surface comprises a substantially regular polymer surface interspersed with ceramic granules comprising substantially irregular outer surfaces.

28. The surgical implant of claim 17, wherein the ceramic granules have a diameter of from about 100 μm to about 300 μm.
